(19) United States
(12) Patent Application Publication
(10) Pub. No.: US 2014/0288475 A1
(43) Pub. Date: Sep. 25, 2014

(54) ANKLE-FOOT ORTHOSES

(71) Applicant: Robert John Watts, Fordingbridge Hampshire (GB)

(72) Inventor: Robert John Watts, Fordingbridge Hampshire (GB)

(21) Appl. No.: 14/002,473

(22) PCT Filed: Mar. 1, 2012
(86) PCT No.: PCT/EP2012/053581

§ 371 Date: Apr. 2, 2014

(30) Foreign Application Priority Data
Mar. 1, 2011 (GB) 1103512.8
Oct. 7, 2011 (GB) 1117361.4

(51) Int. Cl. A61F 5/01

(52) U.S. Cl. CPC A61F 5/0111
USPC 602/27

(57) ABSTRACT

An ankle-foot orthosis for resisting plantarflexion of a patient's foot, the orthosis comprising: a first resiliently flexible part (1) that comprises a first portion (3) and a second portion (5) set at an angle to said first portion (3), said first portion (3) being configured to lie against and along a portion of a patient's shin in use, and said second portion (5) being configured to extend partway along a dorsal aspect of the patient's foot in use, said first part (1) further comprising means (9) operable to secure said first portion (3) against the patient's shin; and a second resiliently flexible part (7) that is separate from but co-operates with said first part (1) in use, said second part (7) comprising a tubular body that is configured and arranged to fit around the patient's foot and to overlap at least part of the second portion (5) of said first part (1); wherein said first (1) and second parts (7) co-operate to provide a resistance to flexure suitable for correcting the plantarflexion experienced by the patient.

### FIELD OF THE INVENTION

This invention relates, in general terms, to ankle-foot orthoses. One preferred embodiment of the invention relates to an ankle-foot orthosis, and another embodiment relates to a method of fitting an ankle-foot orthosis to a patient.

### BACKGROUND TO THE INVENTION

Orthoses are mechanical devices which impose forces upon a limb of a patient and which can be used for a variety of different purposes. For example, orthoses can be provided for supportive, functional, corrective or protective purposes, or indeed for a combination of these.

Ankle-foot orthoses are typically used to provide support against excessive plantarflexion, or “foot-drop” as it is more colloquially known. Foot plantarflexion is a medical condition that results from disease, trauma or congenital abnormality. Patients affected by the condition typically experience difficulty in walking as their feet tend to drop when lifted off the ground, and to avoid stumbling they typically have to lift their foot higher than they would otherwise have to. It is also not atypical for patients to have problems during the swing-through phase of their gait cycle, as a typical sufferer will tend also to exhibit poor, or impaired, dorsiflexion.

The primary function of an ankle-foot orthosis is to provide a resistance to plantarflexion which helps keep the patient's foot in the correct position when the foot is lifted off the ground. As well as this resistive function, a good ankle-foot orthosis should also provide a degree of assistance to dorsiflexion during the swing-through phase of the patient's gait.

A variety of different ankle-foot orthoses have previously been proposed for resisting plantarflexion, and in some cases for additionally assisting dorsiflexion.

One previously proposed device is commonly known in the art as an “under foot” orthosis. As this colloquial name suggests, the orthosis fits under the foot, and in this case outside of a shoe. This particular device cannot be worn without a shoe, and as such the shoe is an integral component of the orthosis. The orthosis comprises a pair of supporting metal rods, one connected to either side of the shoe in the region of the heel by means of a plantarflexion stop that prevents further foot drop. The upper ends of the rods are connected to a supporting band which is secured about the calf of a patient.

Another previously proposed “under foot” orthosis (which must also be used with a shoe) comprises a rigid one-piece plastics moulding composed of integral sole-abutting and calf-abutting regions. The top of the calf-abutting region is provided with a closure mechanism that enables the device to be secured to the calf of a patient, and the sole abutting region acts in conjunction with the shoe to support the foot of the patient.

Another previously proposed device is known colloquially as an “over foot” orthosis, meaning that the orthosis fits over the front (dorsal) aspect of the foot, rather than under it as in the abovementioned previously proposed devices. This “over foot” orthosis comprises a rigid plastics shell which is worn up against the shin, and which is secured around the calf by means of an appropriate securing band. The orthosis includes a stirrup which fits over the foot in the region of the instep to provide the patient with a resistance to plantarflexion.

All of the aforementioned orthoses provide the patient with a device which is capable of resisting plantarflexion. However, it is also the case that each of them has a number of attendant disadvantages.

To alleviate these problems, we have previously provided (see granted UK Patent No. 2330309) a sock-like structure which is formed of a resiliently flexible material—such as silicone for example. The sock-like structure, by virtue of the inherent resilience of the material from which it is made, provides a resistance to plantarflexion and also stores energy which can subsequently be released to assist dorsiflexion. The orthosis can be coloured to mimic the colour of the patient's' skin (and as such is cosmetically pleasing), can comfortably be worn in a normal off-the-shelf shoe, and need not be worn with a shoe in order to provide a beneficial effect.

The part of the sock-like structure which envelops the patient's ankle and lower leg in use includes an opening (to permit the user to put on the device), and in the preferred arrangement the opening is closed (to secure the orthosis in place on the foot of a patient) by means of respective parts of a mechanical hook and loop closure (such as velcro™) which are embedded in the sock-like structure. Typically, one part of the closure is provided on the outside of the orthosis adjacent one side of the opening and the other part is provided on the inside surface of a tab extending from the other side of the opening, the closure on the tab being attachable to the closure adjacent the one side of the opening to close the opening, and secure the orthosis in place.

Our previous orthosis represented a quantum leap in the field and alleviated most (if not all) of the disadvantages mentioned above (the bulk of which had long been associated with previously proposed devices). Whilst this orthosis has proved to be extremely commercially successful, it remains the case that the process by which such devices are manufactured and fitted is relatively time consuming, and as a result these devices are more expensive to purchase than we would otherwise like.

To address this problem we have previously proposed, in United Kingdom Patent Application No. 2420716 for example, to provide a different arrangement for closing the opening in the part of the sock-like structure that envelops the patient's ankle and lower leg in use. Specifically, in the preferred embodiment described in this application, closure of the opening is achieved by configuring the part of the orthosis that envelops the lower leg and ankle of the patient so that it includes a second part that is arranged to overlap a first part when the orthosis is in a closed position fitted about the lower leg of the patient (the second part being moveable in a direction away from the first part to open the opening). One or more discrete closures are then fixedly attached (for example by a so-called speed rivet) to the second part and these closures can be wound round the lower leg of the patient and then secured to themselves to keep the opening closed in use.

One advantage of this arrangement is that by providing one or more separate closures, the embedding of Velcro™ in a silicone body can be avoided, and hence the ankle-foot orthosis can more quickly and inexpensively be manufactured than the orthosis described in our previous UK patent GB2330309.

Whilst the orthosis of GB2420716 does greatly improve upon the orthosis described in GB2330309 (at least from the point of view of the time required to manufacture and fit the orthosis, and hence the cost of purchasing the orthosis), the process of making and fitting such orthoses is still relatively lengthy, and as a consequence devices of the type described in this application are still relatively expensive—particularly for those patients who do not have medical insurance or who are not eligible for state support.

To address these issues we have previously proposed, in GB2456544 to provide an ankle-foot orthosis that can more quickly be manufactured and fitted to a patient (as compared for example with the orthosis described in GB2330309). Specifically, by providing an arrangement where the first portion of the resiliently flexible structure can be fitted round the foot of a patient and then secured in place, it may no longer be necessary to cast each patient's foot before fitting. Instead one can simply select a suitable resiliently flexible structure from a previously manufactured stock of different resiliently flexible structures (each of which is suitable for patients whose feet fall within a relatively small range of foot sizes, for example up to three shoe sizes, and for whom the resistance to plantarflexion provided by the resiliently flexible structure is suitable for correcting the particular degree of plantarflexion that they experience) and then adjust that selected structure to provide an orthosis that fits the patient. It is also the case that manufacture of such a structure is generally easier to accomplish than manufacture of a bespoke orthosis (where greater care must be taken to ensure that all measurements taken for the patient's foot are faithfully reproduced), and hence the process by which such a structure is manufactured is generally faster than that for a wholly bespoke orthosis. The devices disclosed in GB2456544, GB2330309 and GB2420716 have been commercially successful, and have been extremely popular with patients. One by-product of this is that it is often difficult to manufacture these devices in sufficient quantities to satisfy the demand for such products. As a result patients presenting with plantarflexion may sometimes have to wait several weeks for their bespoke device to be manufactured and then fitted.

As will be apparent, whilst a given patient is waiting for their orthosis to be manufactured, they will still be afflicted by the problems that prompted them to seek assistance in the first place, and it would be desirable if a device could be devised that could very quickly be fitted to most patients presenting with excessive plantarflexion, even if such devices were only used by patients as a temporary measure whilst they wait for their customised orthoses to be manufactured and fitted.

Another problem associated with these previously proposed orthoses is the fact that making the determination of the degree of resistance to plantarflexion that the orthosis must provide to correct the particular degree of plantarflexion experienced by a given patient is a relatively difficult task, even for highly skilled and experienced clinicians.

A first step in making that determination usually involves the clinician visually assessing the gait of the patient, and then using their experience to estimate the degree of resistance to plantarflexion (provided by the resilience of the orthosis) that an orthosis will require if it is to correct the particular degree of plantarflexion exhibited by the patient. For devices such as those disclosed in GB2330309 and GB2420716, once the degree of resistance to plantarflexion has been estimated the clinician typically takes a cast of the patient's foot and lower leg, following which an orthosis having the previously determined resistance is formed on the cast. The patient is then required to return to the clinician to try the new orthosis whilst the clinician observes the gait of the patient and determines if the degree of resistance provided by the orthosis needs to be adjusted. This iterative process continues until the orthosis provided to the patient is suitable for the task at hand, and it will be appreciated that the length of time required to complete that process will depend to a large extent on the accuracy of the clinician's initial assessment of the resistance to plantarflexion that the orthosis should provide.

Whilst the device disclosed in GB2456544 helps to facilitate the process whereby orthoses are manufactured (principally by removing the need to make a cast of the patient's foot and lower leg), it will be apparent that the overall length and complexity of the production process will still depend in no small part on the accuracy of the clinician's initial assessment of the resistance to plantarflexion that an orthosis for a given patient should provide.

Whilst many clinicians fitting orthoses have sufficient experience to make accurate assessments, it would nevertheless be advantageous—for example for those clinicians with less experience—to provide an arrangement that facilitated the determination of the required degree of resistance to plantarflexion that an orthosis should provide to assist a given patient.

The present invention has been devised with the foregoing in mind.

### SUMMARY OF THE INVENTION

In accordance with an envisaged implementation of the teachings of the invention, there is provided an ankle foot orthosis for resisting plantarflexion of a patient's foot, the orthosis comprising: a first resiliently flexible part that comprises a first portion and a second portion set at an angle to said first portion, said first portion being configured to lie against and along a portion of a patient's shin in use, and said second portion being configured to extend partway along a dorsal aspect of the patient's foot in use, said first part further comprising means operable to secure said first portion against the patient's shin; and a second resiliently flexible part that is separate from said first part and co-operates with said first part in use, said second part comprising a tubular body that is configured and arranged to fit around the patient's foot and to overlap at least part of the second portion of said first part; wherein said first and second parts co-operate to provide a resistance to flexure suitable for correcting the plantarflexion experienced by the patient.

As is described in detail hereafter, the orthosis of this arrangement can quickly and easily be fitted to patients presenting with plantarflexion, and hence can readily be employed as a temporary means for treating that condition—for example whilst the patient has a bespoke device made for them.

In one arrangement, said first part is generally L-shaped in longitudinal cross-section, and C-shaped in lateral cross-section.

In a preferred implementation at least one of said first part and said second part are of silicone elastomer. The first part and the second part could both be of silicone elastomer.

The resilience of said second part may be greater, smaller or the same as that of said first part. The first part may be of 50 to 90 shore silicone elastomer, The second part may be of 10 to 30 shore silicone elastomer. The first part may be of 70 shore silicone elastomer, and said second part may be of 20 shore silicone elastomer.

The closure may comprise a strap fixedly attached at one end to a lateral edge of said first portion, said strap being configured to be passed round the entire circumference of the patient's lower leg section before being secured to itself to hold the first portion to the patient's shin.

The closure may comprise a second strap fixedly attached at one end to a lateral edge of said first portion, said strap being configured to be passed round the entire circumference of the patient's lower leg section before being secured to itself to hold the first portion to the patient's shin.

The strap may be secured to said first part by means of a speed rivet.

The first part may be selected from a group of differently sized first parts, and said second part may be selected form a group of differently sized second parts.

The group of second parts, may in one implementation, be larger in number than said group of first parts.

The second portion of said first part may extend, in use, along the dorsal aspect of the patient's foot to the patient's toes.

The first part may be configured to cover the patient's medial and lateral malleolae. The second part may extend in use from the patient's toes to the patient's medial and lateral malleolae.

In another arrangement, there is provided a method of fitting an ankle-foot orthosis to a patient, the method comprising: selecting a first part that is suitably sized for fitting to the patient, said first part comprising a first portion and a second portion set at an angle to said first portion, said first portion being configured to lie against and along a portion of a patient's shin in use, and said second portion being configured to extend partway along a dorsal aspect of a patient's foot in use, said first part further comprising means operable to secure said first portion against a patient's shin; and selecting, from a group of differently sized second resiliently flexible parts that co-operate with said first part, a second part that is suitably sized for fitting to the patient, said second parts each comprising a tubular body that is configured and arranged to fit around a patient's foot and to overlap at least part of the second portion of a said first part; fitting said selected first part to said patient so that said first portion lies against and along a portion of the patient's shin and said second portion extends partway along the dorsal aspect of the patient's foot; and fitting said selected second part to said patient so that said second part fits around the patient's foot and overlaps at least part of the second portion of said first part, wherein said first and second parts co-operate to provide an ankle-foot orthosis that fits the patient and provides a resistance to flexure that helps correct the plantarflexion experienced by the patient.

In yet another arrangement, there is provided a system for facilitating the fitting of ankle-foot orthoses to patients, the system comprising: a first resiliently flexible part, said first part being of silicone elastomer and comprising a first portion and a second portion set at an angle to said first portion, said first portion being configured to lie against and along a portion of a patient's shin in use, and said second portion being configured to extend partway along a dorsal aspect of a patient's foot in use, said first part further comprising means operable to secure said first portion against a patient's shin; and a group of differently sized second resiliently flexible parts that co-operate with said first part, said second parts each being of silicone elastomer and comprising a tubular body that is configured and arranged to fit around a patient's foot and to overlap at least part of the second portion of a said first part; wherein a first part and a second part selected from said group co-operate to provide an ankle-foot orthosis that fits the patient and provides a resistance to flexure that helps correct the plantarflexion experienced by the patient.

Other features, aspects and advantages of the teachings of the invention are set out hereafter and elsewhere in the present application.

### BRIEF DESCRIPTION OF THE DRAWINGS

Various aspects of the teachings of the present invention, and arrangements embodying those teachings, will hereafter be described by way of illustrative example with reference to the accompanying drawings, in which:

FIG. 1 is a photograph of a first part of an ankle-foot orthosis;

FIG. 2 is a photograph of a second part of the ankle-foot orthosis;

FIG. 3 is a photograph of the first part fitted to a patient;

FIGS. 4 and 5 are photographs showing, in each photograph, first and second parts that have been fitted to a patient to provide an ankle-foot orthosis;

FIG. 6 is a schematic perspective view of another ankle-foot orthosis; and

FIGS. 7 to 9 are schematic representations of a proposed fitting process.

### DETAILED DESCRIPTION OF PREFERRED EMBODIMENTS

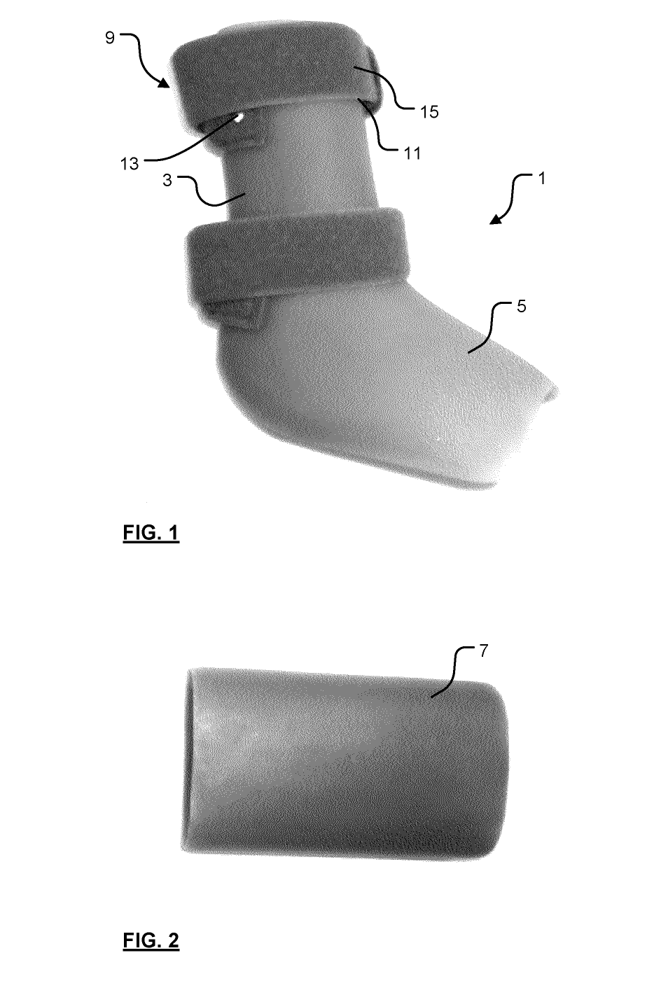

FIG. 1 is a side elevation of a first part 1 of the orthosis according to an illustrative embodiment of the present invention. Whilst the orthosis depicted in FIG. 1 is primarily intended to be used as a temporary measure whilst a bespoke device is being manufactured, it will be apparent that the orthosis could instead be provided as a replacement for a bespoke orthosis. In another particularly preferred implementation, the orthosis depicted in FIG. 1 may be employed as a means to facilitate the determination of the required resistance to plantarflexion that an orthosis should provide to correct the particular degree of plantarflexion experienced by a given patient. In parts of the following detailed description, particular emphasis will be placed on the temporary use of such a device, but it should be remembered that the orthosis depicted has other applications and hence the following detailed description should not be interpreted as being limited only to temporary orthoses.

With this proviso in mind, reference is made to FIG. 1 in which the first part 1 is shown as being generally L-shaped in longitudinal section (i.e. from end to end) and generally c-shaped in lateral cross-section (i.e. from side to side). The first part comprises a first portion 3 and a second portion 5. The first 3 and second 5 portions are contiguous, and in most instances the second tubular portion 5 will have been integrally formed with the first portion 3. The first and second portions are set at an angle to one another and dimensioned so that the first portion lies against and along a portion of a patient's shin in use, and the second portion extends at least partway along a dorsal aspect (i.e. that side of the foot opposite to the sole of the foot) of the patient's foot in use.

In a highly preferred arrangement the first part is formed not so that the first portion is set perpendicular to the second, but so that the structure exhibits approximately 3 to 10, preferably 5, degrees of dorsiflexion. In the preferred arrangement this dorsiflexion drops to about zero when the orthosis is being worn by a patient and the weight of their foot is applied to the orthosis during the swing-through phase of their gait.

In one envisaged arrangement, the second portion extends from the ankle of the patient to the base of their toes (and hence extends along substantially all of the dorsal aspect of the foot), but it will be appreciated that the second portion need not extend all the way along the dorsal aspect of the foot.

In a preferred arrangement, the first part covers both the patient's medial malleolus (the inside of the ankle) and their lateral malleolus (the outside of the ankle), and hence helps protect those parts of the patient. For those patients with particularly sensitive ankles, the first and second portions may be configured so that the walls of the first and second portions are thinned or include apertures in those regions of the first part that would normally overlie the medial and lateral malleolae.

In the preferred arrangement (as depicted) the first part is configured so that the patient's toes and calcaneum (heel) are exposed. This is because we have found that an orthosis which exposes the heel and toes is significantly more comfortable for the patient to wear, whilst also giving the patient a greater sense of confidence when walking (particularly when barefoot) on a given surface due to the fact that their toes and heel can grip that surface.

As aforementioned, the first part is generally C-shaped in cross-section, and as a consequence when the first part is worn by a patient the first portion extends from a first edge that is generally parallel with a notional line running up the lower leg from the lateral malleolus, over the shin of the patient to a second edge that is generally parallel with a notional line running up the lower leg from the medial malleolus.

The first part 1 of the orthosis co-operates with a second part 7, depicted in FIG. 2, to provide an ankle-foot orthosis that resists plantarflexion of a patient's foot. The second part 7 comprises a resiliently flexible tube that is pulled over the patient's foot so that it overlies at least part of the second portion 5 of the first part 1, and encircles the patient's foot. In a particularly preferred arrangement, the second part, when worn by the patient, overlaps substantially all of the second portion 5, and may also extend to cover at least part of the patient's lateral and medial malleolae. As will be appreciated by persons skilled in the art, the primary function of the second part 7 is as a means for tethering the patient's foot to the second portion 5 of the first part 1.

The first and second parts co-operate, when worn by the patient, to provide an ankle-foot orthosis that is configured to resist excessive plantarflexion. To this end, the resilience of the first part 1 and second part 7 may, in one implementation, be chosen to be sufficiently large to resist the most prevalent degrees of plantarflexion, or in another implementation be chosen in dependence upon the degree to which the patient suffers from plantarflexion. The resilience of the first part may be the same as that of the second part, or in other envisaged arrangements the resilience of the first part may be larger or smaller than that of the second part.

Variations in the resilience of a given orthosis (as may be required for patients experiencing a lesser or greater degree of excessive plantarflexion) may be effected by changing the material from which the orthosis is formed, by changing the thickness of one or more of the component parts of the orthosis, or by incorporating reinforcing means—such as a resilient rib—into one or more parts of the orthosis (or indeed by means of a combination of these).

As an illustrative example, the first part could include reinforcing means in the form of a resilient rib that extends partway along the dorsal aspect of the foot. The rib 17 could be formed integrally with the orthosis, or could be removably insertable into a pocket provided on the dorsal aspect of the orthotic structure. The latter arrangement would be particularly useful in that it would allow for the stiffness of the rib and/or the angle of support to be changed if desired (by inserting differently shaped ribs). The rib may be of the same material as the remainder of the orthotic structure 10, or may be of a different—preferably more resilient—material such as plastics, metal or carbon fibre.

The first and second parts are each of a resiliently flexible material, and in the preferred arrangement are each formed of silicone elastomer. In one envisaged implementation, the first part is formed of silicone elastomer having a greater resilience than that of the second part. For example, the first part may be of 50 to 90 shore silicone elastomer, and the second part may be of 10 to 30 shore silicone elastomer. In a particularly preferred arrangement, the first part is of 70 shore silicone elastomer, and said second part is of 20 shore silicone elastomer.

The use of a resilient material for the orthosis (and optionally for the rib, if provided) is a fundamental departure from “under foot” or “over foot” devices of the type described above. These previously proposed devices recommended the use of a non-resilient, i.e. rigid, material. The primary advantage of using a resilient material as opposed to a rigid material is that the material can flex to store energy during certain phases of the patient's gait, and then release that energy during other phases of the patient's gait (in particular the swing-through phase) to thereby actively assist the walking process as a whole, and dorsiflection in particular.

Dorsiflexion and plantarflexion of a foot is predominantly controlled by the tibialis anterior muscle and tendon, and the structure of the orthosis functions to assist the operation of this muscle—in particular for those patients who experience persisting foot drop resulting from a neurological impairment caused, for example, by trauma, disease or genetics. This bio-mechanical function of the device of the invention is fundamentally different to so-called athletic support stockings, for example those of the tubigrip™ type, which provide no means for assisting the operation of the tibialis anterior muscle and tendon (to resist plantarflexion and assist dorsiflexion of a patient's foot), and are instead wholly concerned with resisting abnormal lateral movement of the foot.

Referring now to FIG. 1, the first part 1 comprises a closure 9 that can be opened to allow the patient to place the first part against their shin. The closure 9, in one envisaged arrangement, comprises a first (and optionally a second) strap 11 that is fixed at one end of one lateral edge of the first portion 3 of the first part The strap may, for example, be fixed to the first portion 3 by means of a fixing 13 such as a tubular double-headed rivet (colloquially known as a “speed rivet”), the like of which are available from Evans and Evans (Unit 24, Red Lion Business Park, Red Lion Road, Tolworth, Surbiton, Surrey, UK), or Algeos (Sheridan House, Bridge Industrial Estate, Speke Hall Road, Liverpool, L249HB United Kingdom). Whilst it is undoubtedly the case that a variety of different fixings will be immediately evident to those persons skilled in the art, speed rivets are nevertheless preferred as they permit the attachment of the closure to the orthosis to be accomplished quickly.

The strap 9 carries, on its outer surface 15 (i.e. the surface visible in FIG. 1) a first part of a two-part complementary mechanical engagement mechanism (such as Velcro™), and an end portion of the strap 9 distal from the fixed one end of the strap is provided with a region (not shown) on an inner surface of the strap 9 that includes the second part of the aforementioned two-part engagement mechanism. In a particularly preferred arrangement, the outer surface 15 of the strap carries a series of loops that can be engaged by hooks carried on the inner surface. This arrangement is preferred as the hooks are then less likely to snag on clothes worn by the patient.

Once the first part has been positioned with the first portion lying against the patient's shin and the second portion extending along at least part of the dorsal aspect of the foot, the strap is wound behind the patient's leg, round the front of the first portion and then secured to itself to hold the first portion firmly against the patient's shin. A second strap, if provided, is similarly secured.

Once the strap or straps have been secured around the patient's lower leg, the second part 7 is then pulled over the foot so that it overlaps at least part of the second portion 5 of the first part, whereupon the second part holds (by virtue of its inherent resilience) the second portion tightly against at least part of the dorsal aspect of the patient's foot. When so configured, the first and second parts co-operate to provide an ankle-foot orthosis that resists abnormal plantarflexion of the patient's foot.

Whilst the above arrangement is preferred, it will be apparent that a number of alternative closure mechanisms could instead be provided without departing from the scope of the present invention. For example, the closure could be removable from the orthosis and/or the mechanical hook and loop closure mechanism could be replaced by another type of fixing—such as press studs for example.

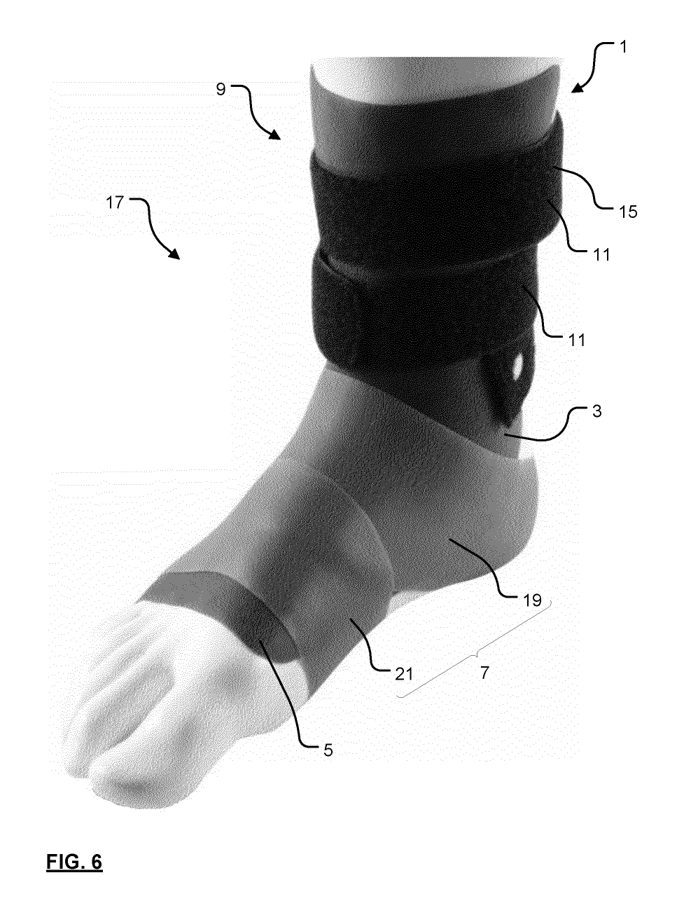

FIG. 6 is a schematic perspective view of an ankle-foot orthosis 17 according to another embodiment of the present invention. In this embodiment, the first part 1 is the same as in the first embodiment described above, and for brevity will not be further described.

The unitary second part 7 of the first embodiment comprises, in this second embodiment, a plurality of resiliently flexible tubes. In a preferred arrangement the second part 7 comprises a first and a second resiliently flexible tube 19, 21 that are each pulled over the patient's foot so that they overlie at least part of the second portion 5 of the first part 1, and encircle part of the patient's foot. In this embodiment of the invention the first tube covers the lateral and medial malleolae and at least part of the calcaneum of the patient, whereas the second tube encircles the foot in the region of the instep.

The first tube 19 functions to resisting “gapping” that can sometimes occur with the orthosis of the first embodiment, particularly in the dorsiflexion phase of a patient's gait. When gapping occurs the first part 1 (particularly in the vicinity of the junction between the first and second portions 3, 5) can tend to come away from the patient's foot and leg, and in the second embodiment the first tube 19 functions to draw the first part (particularly in the vicinity of the junction between the first and second portions 3, 5) towards the patient's foot and lower leg, thereby reducing the likelihood of gapping occurring.

In one envisaged arrangement, the first tube 19 is fitted onto the patient's foot before the second tube 21, and then the second tube 21 is fitted so as to overlie both the second portion 5 of the first part 1 and part of the first tube 19. It will be appreciated, however, that the second tube 21 could instead be fitted before the first 19.

The first tube 19 is, in one implementation, wider than the second tube 21. That is to say, if both the first and second tubes were to be stood on a planar surface (with an open end of each tube abutting the surface) then the first tube would be taller than the second. In other implementations, the second tube may be wider than the first, or may the same width as the first. The first and second tubes 19, 21 may have the same diameter, or different diameters. In one envisaged implementation the first tube 19 has a greater diameter than the second tube 21.

The first and second tubes 19 may have the same resilience or may have different resiliences. As will be appreciated, by providing that the resilience of these tubes may be different, technicians fitting such devices to patients are provided with the ability to better tailor the resistance to plantarflexion provided by the orthosis as a whole to the particular requirements of a given patient.

In an envisaged implementation of the teachings of the invention, a person fitting orthoses will be provided with a kit consisting of a plurality of differently sized first and second parts (which second parts may comprise a single tube (as in the first embodiment), or more than one tube (as in the second embodiment). That person then need only select appropriately sized first and second parts for a given patient, and hence that patient can quickly be provided with an ankle-foot orthosis that corrects plantarflexion—which orthosis could be utilised temporarily whilst a bespoke silicone ankle-foot orthosis (for example of the type described in GB2456544, GB2330309 or GB2420716) is manufactured. The kit could contain differently sized first parts and second parts that are, respectively, of the same resilience (i.e. of a resilience appropriate for correcting more prevalent degrees of plantarflexion), or the kit could be expanded to include first and second parts of different sizes and resiliences. In this latter arrangement, the resulting orthosis could more easily be tailored to provide a resistance that is appropriate for addressing the particular degree of abnormal plantarflexion experienced by a given patient.

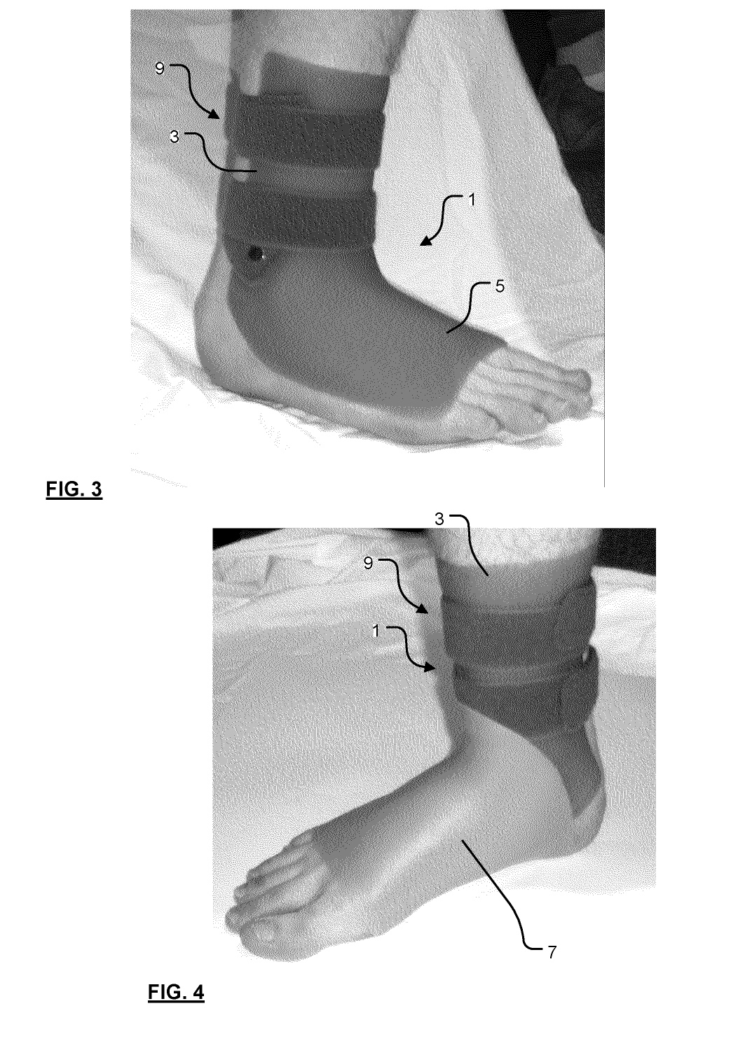

In circumstances where the kit contains first and second parts that have, respectively, the same resilience, a person fitting the orthosis need only select a first part that is appropriately sized for the particular patient they are seeing at the time. Once a suitable first part has been identified, the first portion is placed against the patient's leg so that the second portion extends along at least part of the dorsal aspect of the patient's foot (as shown in FIG. 3), following which the strap(s) 9 are secured around the patient's leg to hold the first part in place.

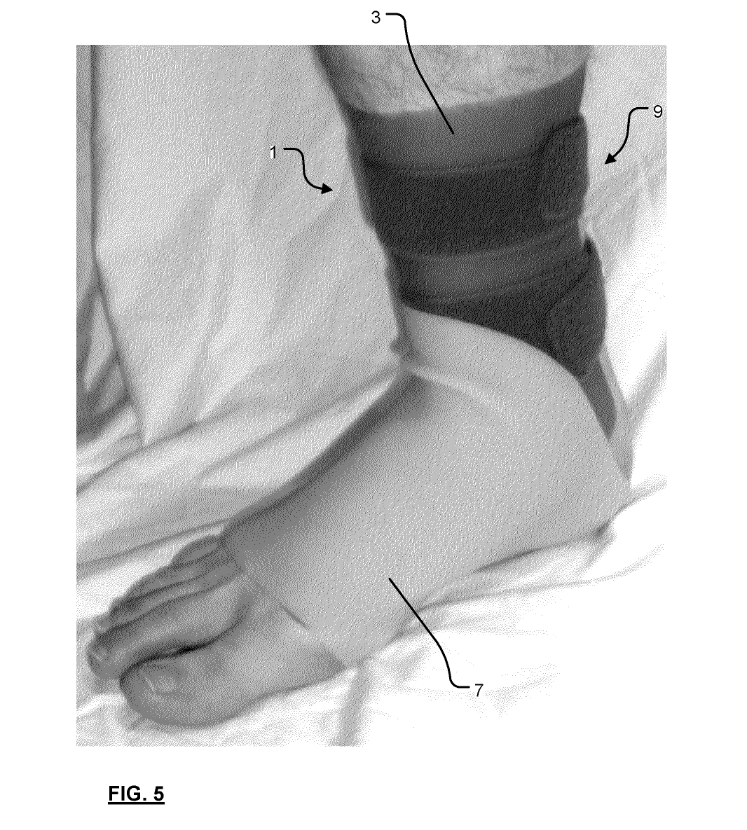

Next, the person fitting the orthosis need only select an appropriately sized second part from the kit and pull that second part over the patient's foot so that it overlaps at least part (and preferably all) of the second portion of the first part. Once fitted in this position, as shown in FIGS. 4 and 5, the patient is provided with an ankle foot orthosis that is operable to resist plantarflexion.

In circumstances where the kit contains components of various resiliences, the person fitting the orthosis should select first and second parts that are both appropriately sized, and have an appropriate resilience to resist the particular degree of abnormal plantarflexion experienced by the patient to whom the orthosis is being fitted.

It is envisaged that most patients can be accommodated with a kit that contains a relatively small number of differently sized first and second parts. For example, in one implementation the kit comprises five differently sized second parts and three differently sized first parts, and it can be seen from the foregoing that the process of fitting such devices to patients can quickly and easily be accomplished.

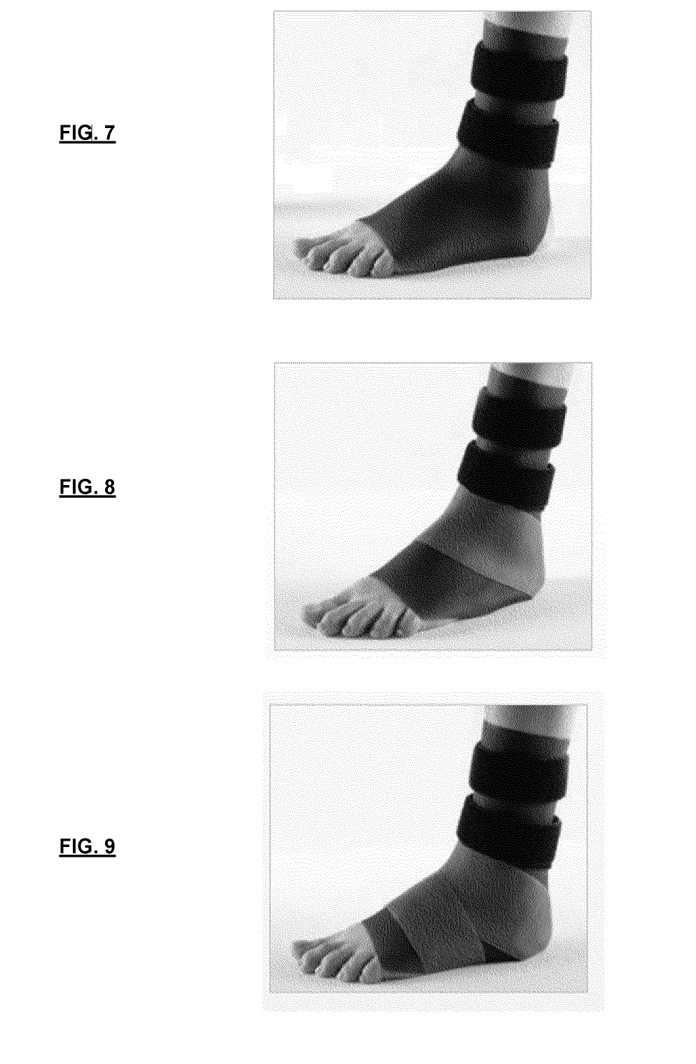

FIGS. 7 to 9 illustrate this process for the orthosis depicted in FIG. 6. In a first step of the process the clinician selects a first part that has a resilience which is appropriate for addressing the particular degree of plantarflexion that the clinician has observed in a given patient. The clinician then selects a first tube that is of an appropriate resilience and size for the patient and fits that tube over the first part. The clinician then selects a second tube that is of an appropriate resilience and size for the patient, and first that second tube over the first part and the first tube.

A particular advantage of the arrangements described herein is that the accuracy of a clinician's initial assessment of the resistance to plantarflexion that is required for a given patient becomes less critical, as the clinician can easily and quickly vary the resilience of the orthosis as a whole by changing one or more parts of the orthosis for a substitute part having a greater or lesser resilience. Thus with a system such as that described herein, a clinician has to rely to a lesser extent on their experience as they can easily and quickly swap components until they have an orthosis that is appropriate for the particular degree of plantarflexion experienced by a given patient.

The devices described herein may be coloured so that they can be matched to the skin colour of the patient (as can the closure member(s)) or indeed to have any other colour. The first and second parts could even be brightly and/or differently coloured to improve their appeal to children. The resiliently flexible structure could be manufactured by injection moulding (or any other suitable process), but in the preferred embodiment it is manufactured by milling (as described below) and subsequently building up layers of material, for example silicone elastomer, upon a suitably sized generic three dimensional representation of a foot and lower leg.

The resiliently flexible structure, as mentioned above, may be manufactured from a number of different resiliently flexible materials. Amongst these, silicone elastomer is a particularly preferred material. Two suitable elastomers are sold under the product names HCR9960 and MED4035 by Nusil Technology of 1050 Cindy Lane, Carpinteria, Calif., USA. Another family of suitable elastomers are sold by Nusil Technology under the registered trademark VersaSil3.

HCR9960 has a working time of approximately 12 hours and MED4035 has a shorter working time of approximately 3 to 4 hours, after which the elastomer cures. The elastomers are thermo-setting and are strained through a 200 mesh screen to remove particulate contaminants. The elastomers are supplied as A and B components which are preferably combined in equal portions on a two roll mill, or other suitable device, prior to use.

A suggested sequence for blending the two components is to first soften part B on the mill and then soften part A, after which an equal weight of part B should be added to part A and then thoroughly mixed. At this stage, it is recommended to keep the temperature of the material as low as possible so as to maximise the table life of the elastomer. The mixture may then supplied to injection moulding apparatus to mould a suitably shaped orthotic structure, or more preferably may be manually fitted to a generic three-dimensional representation of a foot and lower leg. Curing of the blended elastomer may be accelerated by heat and can take from 3 to 4 hours. The cure may be inhibited by any ambient traces of organic rubbers and other substances and thus it is important for the fabrication of the orthotic structure to be conducted in a thoroughly cleaned area.

The VersaSil3 family of elastomers include 3 base stocks which when vulcanised produce tough, durable elastomers with Shore A durometers of 30, 50 and 70, and the base stocks can be blended to produce elastomers of intermediate durometer. The three base stocks are compounded with CAT-40 and CAT-55-CAT—being an inhibitor, and CAT-55 being a platinum catalyst.

Each series (i.e. 30, 50 or 70 durometer) is supplied as a three part system which must be compounded, for example on a two roll mill, prior to use. Elastomers of intermediate durometer can be produced by blending 30, 50 or 70 durometer elastomers in a 1:1 ratio. For example, a 40 durometer elastomer can be achieved by blending VersaSil-30 and VersaSil 50 in a 1:1 ratio mix.

To produce a given elastomer it is suggested to soften approximately 25% of the total required base stock on a cooled two roll mill. The entire required quantity of CAT-40 should then be added, and the resulting mixture milled until homogeneous.

Whilst the base/CAT-40 mixture is turning on the mill, small increments of CAT-55 should be added until the entire required amount has been added. Next the remaining base stock should added and milled. Once the elastomer has been produced, the mixture may then be supplied to injection moulding apparatus to mould a suitably shaped orthotic structure, or more preferably be manually fitted to a generic three dimensional representation of a foot and lower leg.

Cure of the resulting mixture is accelerated by heat. For example, the elastomer will cure in a mould cross-section up to 0.075 inch (0.00 195 m) thick in less than ten minutes at 116 C. The vulcanisation rate can be increased by increasing the cure temperature, and an optional post cure, such as four hours at 177 C may be implemented if desired. An important point to note is that cure of the elastomer may be inhibited by traces of amines, sulphur, nitrogen oxide, organo-tin compounds and carbon monoxide. As such it is important for manufacture of the orthotic structure to be conducted in a thoroughly clean environment.

The performance of the device described herein is expected to be similar to that provided by our previously proposed device as described in granted UK Patent No. 2330309. This device was found to provide a considerable improvement not only to the degree of plantarflexion, but also to the walking speed and the effort involved in walking (known as the Physiological Cost Index or PCI) of a group of patients. At an initial point in the study it was found that the orthosis provided an increase of roughly 10% in walking speed and a reduction of roughly 2% in the PCI.

At the end of the study, roughly six months later, it was found that that same group of patients experienced an increase in walking speed of roughly 20% and a reduction of roughly 32% in the PCI as compared to when they were initially without the orthosis.

It is anticipated that the orthosis of the present invention will provide comparable results.

We have also found that orthoses of the type described herein can improve the patient's proprioception. In particular, when not wearing an orthosis as herein described, we have noted that patients often tend to stumble when walking. However, when the orthosis is worn, the pressure exerted by the orthosis on the skin receptors sends a message to the brain that helps the patient determine where the foot is in space. This in turn helps the patients to walk faster and avoid stumbling.

It will be understood from the above that the orthoses herein described provide an effective means to tackle the problem of plantarflexion. Advantageously, and in addition to this function, the orthoses herein described can significantly augment dorsiflexion during the swing-through phase of a patient's gait cycle. The principal reason for this is believed to be that the orthosis (and resilient rib, if provided) stores energy when compressed, and this energy is released during the swing through phase of the patient's gait cycle. It is anticipated, therefore, that patients will not only find that the orthoses tackle the problem of plantarflexion but also actively assist the walking process.

Whilst various illustrative arrangements have been described above in detail, it will be apparent and should be noted that modifications and alterations may be made to those arrangements without departing from the spirit and scope of the invention as defined by the accompanying claims.

It should also be noted that whilst the accompanying claims set out particular combinations of features described herein, the scope of the present invention is not limited to the particular combinations hereafter claimed, but instead extends to encompass any combination of features herein disclosed.

1. An ankle-foot orthosis for resisting plantarflexion of a patient's foot, the orthosis comprising:
a first resiliently flexible part that comprises a first portion and a second portion set at an angle to said first portion, said first portion being configured to lie against and along a portion of a patient's shin in use, and said second portion being configured to extend at least partway along a dorsal aspect of the patient's foot in use, said first part further comprising means operable to secure said first portion against the patient's shin; and
a second resiliently flexible part that is separate from and co-operates with said first part, said second part comprising a tubular body that is configured and arranged to fit around the patient's foot and to overlap at least part of the second portion of said first part;
wherein said first and second parts are of silicone elastomer and co-operate to provide a resistance to flexure that is suitable for correcting the particular degree of plantarflexion experienced by the patient.

2. An orthosis according to claim 1, wherein said first part is generally L-shaped in longitudinal cross-section.

3. An orthosis according to claim 1, wherein said second part comprises a plurality of resiliently flexible tubes.

4. An orthosis according to claim 3, wherein said second part comprises a first and a second resiliently flexible tube, wherein said first and second tubes are of the same or different size and/or resilience.

5. An orthosis according to claim 1, wherein the resilience of said second part is greater, smaller or the same as that of said first part.

6. An orthosis according to claim 5, wherein said first part is of 50 to 90 shore silicone elastomer.

7. An orthosis according to claim 5, wherein said second part is of 10 to 30 shore silicone elastomer.

8. An orthosis according to claim 5, wherein said first part is of 70 shore silicone elastomer, and said second part is of 20 shore silicone elastomer.

9. An orthosis according to claim 1, wherein said closure comprises a strap fixedly attached at one end to a lateral edge of said first portion, said strap being configured to be passed round the entire circumference of the patient's lower leg section before being secured to itself to hold the first portion to the patient's shin.

10. An orthosis according to claim 9, wherein the closure comprises a second strap fixedly attached at one end to a lateral edge of said first portion, said strap being configured to be passed round the entire circumference of the patient's lower leg section before being secured to itself to hold the first portion to the patient's shin.

11-13. (canceled)

14. An orthosis according to claim 1, wherein said second portion of said first part extends, in use, along the dorsal aspect of the patient's foot to the patient's toes.

15. An orthosis according to claim 1, wherein said first part is configured to cover the patient's medial and lateral malleolae.

16. An orthosis according to claim 1, wherein said second part extends in use from the patient's toes to the patient's medial and lateral malleolae.

17. A method of fitting an ankle-foot orthosis to a patient, the method comprising:
selecting a first part that is suitably sized for fitting to the patient, said first part comprising a first portion and a second portion set at an angle to said first portion, said first portion being configured to lie against and along a portion of a patient's shin in use, and said second portion being configured to extend partway along a dorsal aspect of a patient's foot in use, said first part further comprising means operable to secure said first portion against a patient's shin; and
selecting, from a group of differently sized second resiliently flexible parts that cooperate with said first part, a second part that is suitably sized for fitting to the patient, said second parts each comprising a tubular body that is configured and arranged to fit around a patient's foot and to overlap at least part of the second portion of a said first part;
fitting said selected first part to said patient so that said first portion lies against and along a portion of the patient's shin and said second portion extends partway along the dorsal aspect of the patient's foot; and
fitting said selected second part to said patient so that said second part fits around the patient's foot and overlaps at least part of the second portion of said first part, wherein said first and second parts co-operate to provide an ankle-foot orthosis that fits the patient and provides a resistance to flexure that helps correct the plantarflexion experienced by the patient.

18. A system for facilitating the fitting of ankle-foot orthoses to patients, the system comprising:
a first resiliently flexible part, said first part being of silicone elastomer and comprising a first portion and a second portion set at an angle to said first portion, said first portion being configured to lie against and along a portion of a patient's shin in use, and said second portion being configured to extend partway along a dorsal aspect of a patient's foot in use, said first part further comprising means operable to secure said first portion against a patient's shin; and
a group of differently sized second resiliently flexible parts that co-operate with said first part, said second parts each being of silicone elastomer and comprising a tubular body that is configured and arranged to fit around a patient's foot and to overlap at least part of the second portion of a said first part;
wherein a first part and a second part selected from said group co-operate to provide an ankle-foot orthosis that fits the patient and provides a resistance to flexure that helps correct the plantarflexion experienced by the patient.

19. A system according to claim 18, wherein said system comprises a group of first parts, each first part of said group being differently sized as compared with the remainder.

20. A system according to claim 19, wherein each said first part of said group has a different resilience to that of the other first parts in said group.

21. A system according to claim 19, wherein each said second part of said group has a different resilience to that of the other second parts in said group.

22. A system according to claim 18, wherein one or more of said second parts of said group comprises a plurality of tubular bodies.

23. A system according to claim 22, wherein the tubular bodies that comprise said second part have the same resilience or different resiliences.
